(19) United States
(12) Patent Application Publication
(10) Pub. No.: US 2026/0072010 A1
(43) Pub. Date: Mar. 12, 2026

(54) SYSTEM FOR MONITORING URINE STATE

(71) Applicant: MilliTronic CO., LTD., New Taipei City (TW)

(72) Inventor: YA-CHUNG YU, New Taipei City (TW)

(73) Assignee: MilliTronic CO., LTD., New Taipei City (TW)

(21) Appl. No.: 19/325,828

(22) Filed: Sep. 11, 2025

Related U.S. Application Data
(60) Provisional application No. 63/693,690, filed on Sep. 11, 2024.

(51) Int. Cl. G01N 33/493; G01G 17/04; G01G 19/18; G01G 23/32; H04N 7/18

(52) U.S. Cl. CPC G01N 33/493; G01G 17/04; G01G 19/18; G01G 23/32; H04N 7/183; H04N 7/188

(57) ABSTRACT

A system for monitoring urine in a fluid container, including a hanger, a weight scale, a camera, a controlling unit and a communication module. The hanger is for hanging the fluid container. The weight scale measures weight of the fluid container hung by the hanger. The camera takes an image of the fluid container. The controlling unit controls the camera to take the image of the fluid container. The communication module transmits urine information including at least one of the weight and the image.

### CROSS-REFERENCE TO RELATED APPLICATION

This non-provisional application claims priority under 35 U.S.C. § 119(e) on US provisional Patent Application No(s). 63/693,690 filed on Sep. 11, 2024, the entire contents of which are hereby incorporated by reference.

### BACKGROUND OF THE INVENTION

### 1. Field of the Invention

The disclosure relates to a monitoring system and, in particular, to a system for monitoring the state of urine in a urine bag.

### 2. Description of the Related Art

When caring for patients, it is crucial to monitor their physiological states, including the state of the patient's urine. When a patient is lying in a hospital bed and using a urine bag, doctors and nurses need to regularly check the condition of the urine in the urine bag, including its weight and color, to determine whether the patient's physiological state is normal.

As the number of patients increases, monitoring the state of urine creates a workload burden for medical personnel. Therefore, a more efficient solution is needed to reduce the workload of medical personnel and provide timely monitoring information for their reference.

### BRIEF SUMMARY OF THE INVENTION

One objective of the invention is to provide a system for monitoring the state of urine to reduce the workload of medical personnel and provide timely monitoring information for their reference.

To achieve the above objective, the invention provides a system for monitoring urine in a fluid container, including a hanger, a weight scale, a camera, a controlling unit and a communication module. The hanger is for hanging the fluid container. The weight scale measures weight of the fluid container hung by the hanger. The camera takes an image of the fluid container. The controlling unit controls the camera to take the image of the fluid container. The communication module transmits urine information including at least one of the weight and the image.

In one embodiment, the fluid container may be made of flexible material, and the hanger may be a T-shaped hanger or two spaced-apart hooks suitable for expanding the fluid container.

In one embodiment, the weight scale may include an analog-to-digital convertor to convert the weight measured in analog form to digital signals for the reception of the controlling unit.

In one embodiment, the controlling unit may control the camera to take the image of the fluid container periodically or when the weight change of the fluid container exceeds a threshold.

In one embodiment, the communication module may be a wireless transmission module.

In one embodiment, the system may further include a main housing for accommodating the controlling unit and an arm with one end detachably connected with the main housing and another end rotatably connected with the camera. The arm may be configured to place the camera at the side of the fluid container, and may include a connector for establishing an electrical connection between the camera and the controlling unit. The controlling unit may selectively transmit the image based on a status of the electrical connection of the connector.

In one embodiment, the system may further include a display for showing the weight of the fluid container.

In one embodiment, the system may further include an AI engine for identifying a state of the urine in the fluid container based on the urine information. The AI engine may receive the urine information transmitted from the communication module or may be integrated on the same circuit board with the controlling unit.

In one embodiment, the AI engine may include a trained neural network model for identifying the state of the urine in the fluid container based further on other information. The AI engine may further predict a next possible state of the urine or identify a possible symptom of a patient providing the urine.

The system can timely transmit urine information to the medical personnel. It is very easy to use and can significantly reduce the workload of medical personnel and provides timely monitoring information for their reference.

### BRIEF DESCRIPTION OF THE DRAWINGS

FIG. 1 is a schematic diagram showing the architecture of a system for monitoring the state of urine according to an embodiment of the invention.

FIG. 2 is a schematic diagram showing an example of the sensor according to an embodiment of the invention.

FIG. 3 is a schematic diagram showing an example of the processing device according to an embodiment of the invention.

FIG. 4 shows an example of the AI engine according to an embodiment of the invention.

FIG. 5 shows another example of the AI engine according to an embodiment of the invention.

FIG. 6A shows a system 6 for monitoring the state of urine according to another embodiment of the invention.

FIG. 6B shows a system 6 for monitoring the state of urine according to another embodiment of the invention.

FIG. 6C shows a system 6 for monitoring the state of urine according to another embodiment of the invention.

FIG. 6D shows a system 6 for monitoring the state of urine according to another embodiment of the invention.

### DETAILED DESCRIPTION OF THE INVENTION

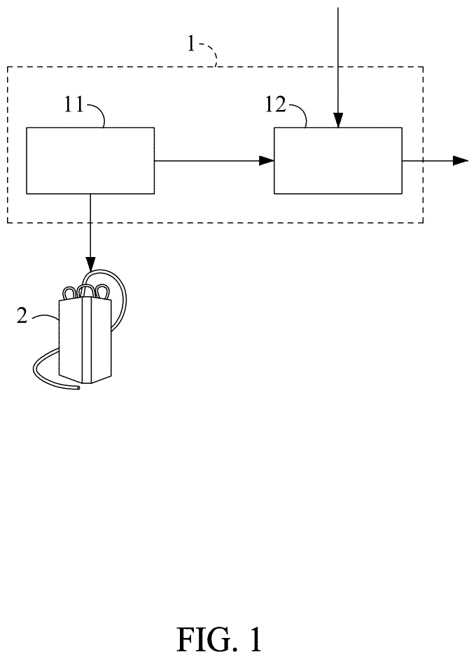

FIG. 1 is a schematic diagram showing the architecture of a system 1 for monitoring the state of urine according to an embodiment of the disclosure. Referring to FIG. 1, the system 1 monitors the state of urine in a fluid container 2, such as a urine bag, and includes a sensor 11 and a processing device 12. The sensor 11 monitors urine in the fluid container 2 and outputs urine information to the processing device 12. The processing device 12 receives the urine information and optionally other information, and outputs a urine state.

The fluid container 2 is designed to collect urine drained from the bladder via a catheter and may be made of flexible and substantially transparent or translucent material such as polyvinyl chloride (PVC), polyvinylidene fluoride (PVDF) or polyethylene. Since fluid container 2 is transparent or translucent, it is possible for the sensor 11 to monitor the color or the turbidity of the urine in the fluid container 2.

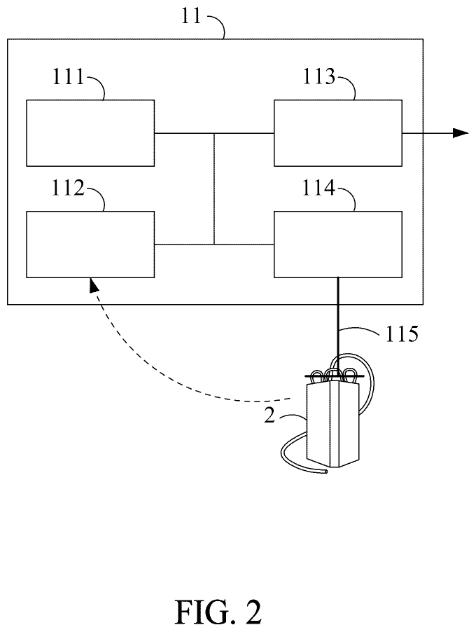

FIG. 2 is a schematic diagram showing an example of the sensor 11. Referring to FIG. 2, the sensor 11 includes an MCU (main controlling unit) 111, a camera 112, a communication module 113, a weight scale 114 and a hanger 115. The MCU 111 may be designed to control the camera 112 to capture an image of the fluid container 2 periodically or when a certain condition is satisfied. The MCU 111 may be an ASIC (application-specific integrated circuit) or the combination of a general-purpose processor and a memory stored with firmware codes for the general-purpose processor to perform specific operations.

The camera 112 may be a digital camera suitable for taking the image of the fluid container 2 and may have an automatic light fill function that automatically fills in light when the ambient light is dark. In practical applications, while the fluid container 2 may be hung below the sensor 11, the camera 112 may be extended from above the fluid container 2 to the side of the fluid container 2 to capture an image from the lateral side of the fluid container 2.

The communication module 113 may be a wireless transmission module such as a Wi-Fi or a Bluetooth module, and wirelessly transmits the urine information to the processing device 12. The urine information may include but not limited to the image and the weight of the fluid container 2. The communication module 113 may also be a wired transmission module such as a USB/Ethernet controller to transmit the urine information via USB/Ethernet protocol.

The weight scale 114 measures the weight of the fluid container 2 and may include an analog-to-digital convertor to convert the measured weight in analog form to digital signals for the reception of the MCU 111. In one example, the MCU 111 may control the camera 112 to capture an image of the fluid container 2 when the weight scale 114 indicates that the weight change of the fluid container 2 exceeds a threshold.

The hanger 115 may be a T-shaped hanger, which is suitable to expand the fluid container 2 made of flexible material. In other implementations, the hanger 115 may be in the shape of a hook or may include multiple clamps to clamp the top of the fluid container 2.

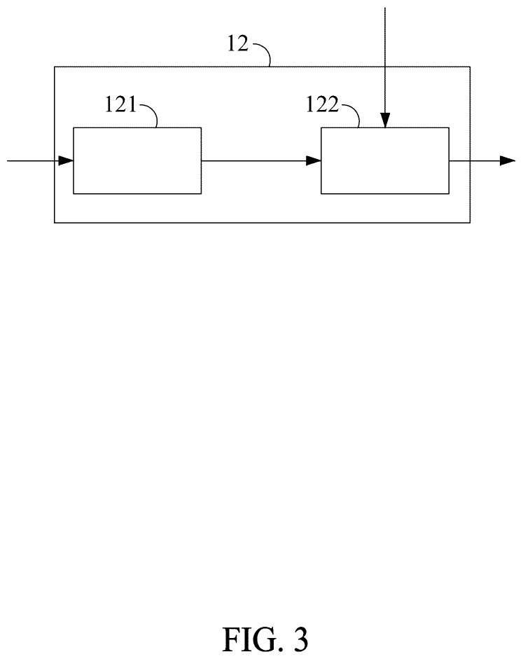

FIG. 3 is a schematic diagram showing an example of the processing device 12, including a communication module 121 and an AI engine 122. The communication module 121 receives urine information from the sensor 11. Similar to the communication module 113 of the sensor 11, the communication module 121 may be a wireless transmission module such as a Wi-Fi or a Bluetooth module or a wired transmission module such as a USB/Ethernet controller.

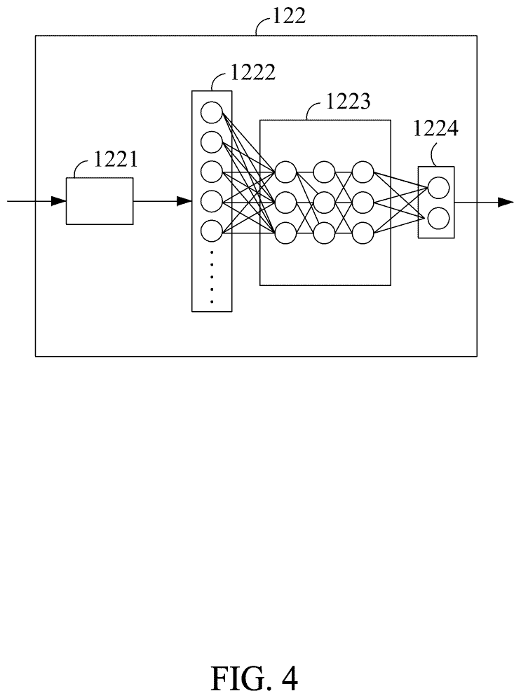

The AI engine 122 may include a neural network model trained to identify the state of the urine in the fluid container 2 based on the urine information. FIG. 4 shows an example of the AI engine 122, including a pre-processor 1221 and a neural network model including an input layer 1222, hidden layers 1223, and an output layer 1224.

The pre-processor 1221 performs normalization, tokenization and vectorization to the urine information. The pre-processed urine information is then input into the input layer 1222 of the neural network model. After being processed by the hidden layers 1223, the output layer 1224 outputs the urine state of the urine in the fluid container 2. The urine state may include, for example, a number indicating whether the urine in the fluid container 2 is normal (indicated by “0”), has hematuria (indicated by “1”), or is turbid (indicated by “2”).

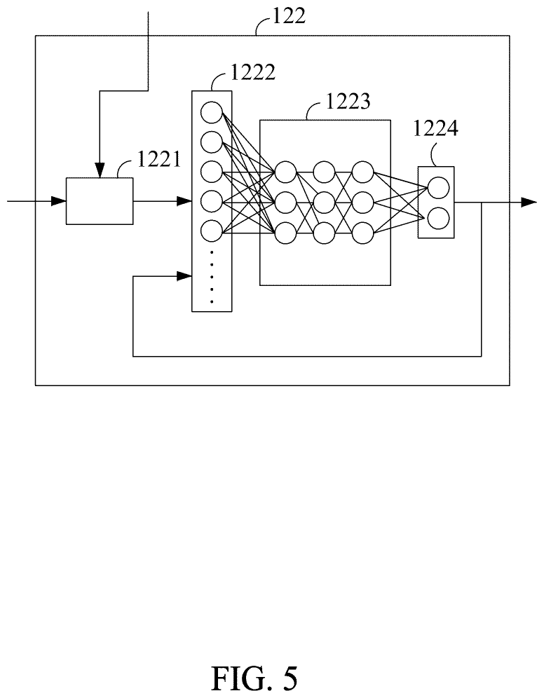

FIG. 5 shows another example of the AI engine 122, in which optional other information are fused with the urine information. The optional other information may include the blood pressure and/or the heartbeat rate of the patient from whom the urine is collected.

In this example, the last output of the output layer 1224 may also be included in the next input of the input layer 1222. This enables the AI engine 122 to predict the next possible state of the urine. For example, the urine state output by the output layer 1224 may also indicate that the urine will be normal (indicated by “4”), will has hematuria soon (indicated by “5”), or will be turbid soon (indicated by “6”).

By fusing the urine information with other information and the last output of the output layer 1224, it is also possible for the AI engine 122 to identify the possible symptom of the patient. For example, it is possible that the output layer 1224 outputs not only the urine state, but also a number indicating a trend for the patient to have bladder inflammation (indicated by “8”) or kidney inflammation (indicated by “9”). This identification of symptoms may be useful for medical personnel caring for patients.

The neural network model in the AI engine 122 may be trained offsite under the supervision of a professional doctor. The professional doctor may use a computer to tag real urine information to prepare an initial training dataset for the neural network model. After an initial training using the initial training dataset, the professional doctor may then perform further supervised training by labeling, for example real urine container images. Once the accuracy of the neural network model reaches an acceptable threshold (such as 99.97%), the trained neural network model is deployed to the processing device 12 to determine the urine state of real patients.

With the above system, the urine state and other possible symptoms of the patient can be monitored efficiently. The system can timely transmit the image of the fluid container 2, the weight of the fluid container 2, and the urine state or possible symptoms determine by the AI engine to the medical personnel. This significantly reduces the workload of medical personnel and provides timely monitoring information for their reference.

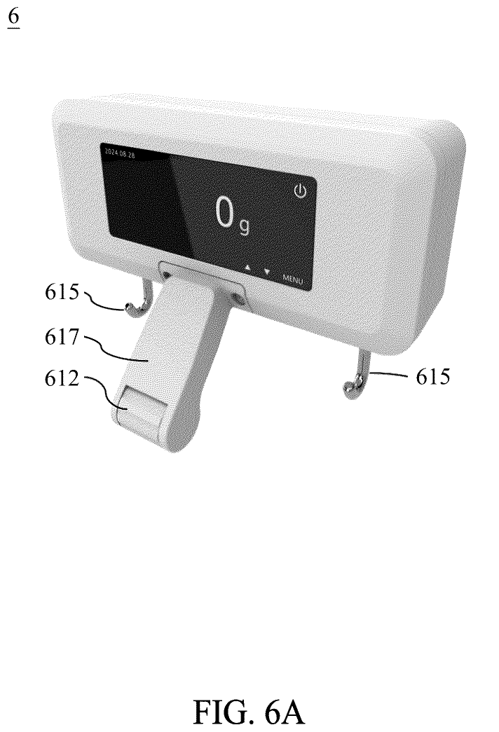

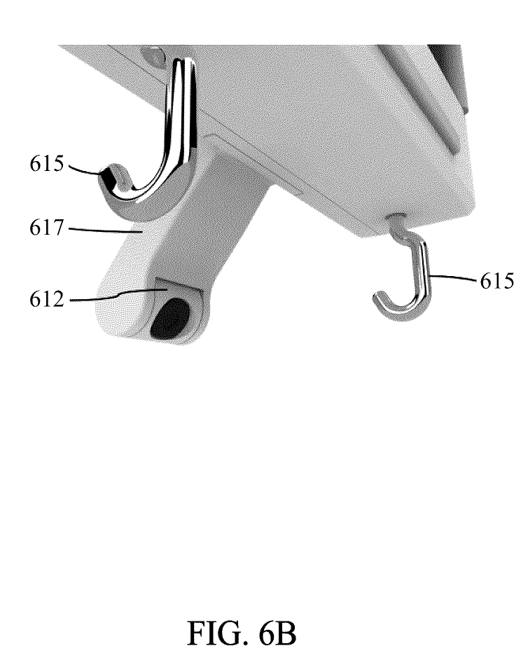

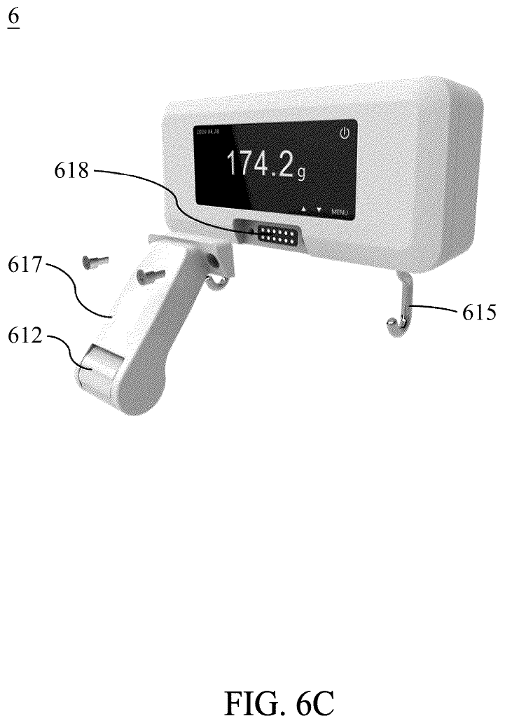

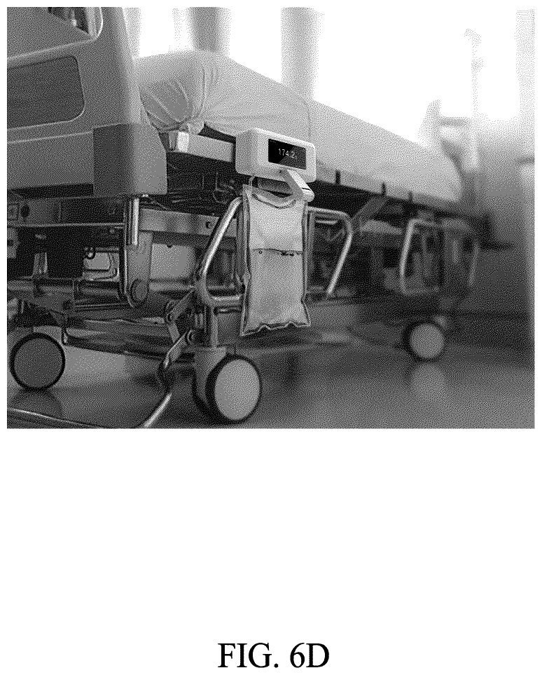

FIGS. 6A to 6D show a system 6 for monitoring the state of urine according to another embodiment of the disclosure. In this embodiment, the system 6 includes two hooks 615 as the hangers for the fluid container 2. The two hooks 615 are disposed at the bottom of the main housing of the system 6 and are spaced apart from each other. One hook 615 is disposed at the right side of the bottom of the main housing of the system 6 and another hook is disposed at the left side of the bottom. This design is suitable to expand the fluid container 2 made of flexible material when the fluid container 2 is hung below the system 6.

The system 6 also has an arm 617. One end of the arm 617 is rotatably connected with a camera 612, while another and of the arm 617 is detachably connected with the main housing of the system 6. The arm 617 is suitable for placing the camera 612 at the side of the fluid container 2 so that the camera 612 is capable of taking an image from the lateral side of the fluid container 2. Since the camera 612 is rotatably connected with the arm 617, it would be convenient for a user to adjust the angle of the camera 612 to capture a better image of the fluid container 2.

The arm 617 is detachably connected with the main housing of the system 6 via two screws. When the arm 617 is connected to the main housing, a connector 618 establishes the electrical and signal connections between the camera 612 and other components in the main housing, such as the MCU. The MCU within the main housing of the system 6 may selectively transmit the image based on the electrical connection status of the connector 618.

In this embodiment, the processing device may be integrated in the same main housing. For that case, the processing device may be integrated on the same circuit board with the MCU of the sensor, and signally connected with the MCU directly via the printed wires of the circuit board. The processing device may also be implemented on a separate board and signally connected with the MCU of the sensor via a flexible circuit board or a board-to-board connector.

The system 6 may also includes a display at the front side of the main housing. The display may show the weight of the fluid container 2 or any information useful for medical personnel when checking the status of the patient. The power button of the system 6 may also be provided at the front side of the main housing for the convenience of the medical personnel.

With the design of this embodiment, the system 6 is suitable for being hang at the side of a bed. The system 6 can timely transmit the image of the fluid container 2, the weight of the fluid container 2, and/or the urine state or possible symptoms determine by the AI engine to the medical personnel. It is very easy to use and can significantly reduce the workload of medical personnel and provides timely monitoring information for their reference.

While the present disclosure has been described by means of specific embodiments, numerous modifications and variations could be made thereto by those skilled in the art without departing from the scope and spirit of the present disclosure set forth in the claims.

What is claimed is:

1. A system for monitoring urine in a fluid container, comprising:
a hanger for hanging the fluid container;
a weight scale for measuring weight of the fluid container hung by the hanger;
a camera for taking an image of the fluid container;
a controlling unit for controlling the camera to take the image of the fluid container; and
a communication module for transmitting urine information including at least one of the weight and the imager.

2. The system for monitoring urine in the fluid container according to claim 1, wherein the fluid container is made of flexible material, and the hanger is a T-shaped hanger suitable for expanding the fluid container.

3. The system for monitoring urine in the fluid container according to claim 1, wherein the fluid container is made of flexible material, and the hanger includes two hooks spaced apart for expanding the fluid container.

4. The system for monitoring urine in the fluid container according to claim 1, wherein the weight scale includes an analog-to-digital convertor to convert the weight measured in analog form to digital signals for the reception of the controlling unit.

5. The system for monitoring urine in the fluid container according to claim 1, wherein the controlling unit controls the camera to take the image of the fluid container periodically.

6. The system for monitoring urine in the fluid container according to claim 1, wherein the controlling unit controls the camera to take the image of the fluid container when the weight change of the fluid container exceeds a threshold.

7. The system for monitoring urine in the fluid container according to claim 1, wherein the communication module is a wireless transmission module.

8. The system for monitoring urine in the fluid container according to claim 1, further comprising:
a main housing accommodating the controlling unit; and
an arm with one end detachably connected with the main housing and another end rotatably connected with the camera.

9. The system for monitoring urine in the fluid container according to claim 8, wherein the hanger includes two hooks disposed at the bottom of the main housing and are spaced apart from each other.

10. The system for monitoring urine in the fluid container according to claim 8, wherein the arm is configured to place the camera at the side of the fluid container.

11. The system for monitoring urine in the fluid container according to claim 8, wherein the arm includes a connector for establishing an electrical connection between the camera and the controlling unit.

12. The system for monitoring urine in the fluid container according to claim 11, wherein the controlling unit selectively transmits the image based on a status of the electrical connection of the connector.

13. The system for monitoring urine in the fluid container according to claim 1, further comprising:
a display for showing the weight of the fluid container.

14. The system for monitoring urine in the fluid container according to claim 1, further comprising:
an AI engine for identifying a state of the urine in the fluid container based on the urine information.

15. The system for monitoring urine in the fluid container according to claim 14, wherein the AI engine receives the urine information transmitted from the communication module.

16. The system for monitoring urine in the fluid container according to claim 14, wherein the AI engine is integrated on the same circuit board with the controlling unit.

17. The system for monitoring urine in the fluid container according to claim 14, wherein the AI engine includes a trained neural network model.

18. The system for monitoring urine in the fluid container according to claim 14, wherein the AI engine identifies the state of the urine in the fluid container based further on other information.

19. The system for monitoring urine in the fluid container according to claim 14, wherein the AI engine further predicts a next possible state of the urine.

20. The system for monitoring urine in the fluid container according to claim 14, wherein the AI engine further identifies a possible symptom of a patient providing the urine.
